(19) United States
(12) Patent Application Publication
(10) Pub. No.: US 2026/0166334 A1
(43) Pub. Date: Jun. 18, 2026

(54) LOWER BACK WEARABLE LIGHT THERAPY DEVICE WITH INTEGRATED VIBRATION ELEMENT

(71) Applicant: PHYSIOHAB LLC, Sunrise, FL (US)

(72) Inventor: Eduardo Marti, Weston, FL (US)

(21) Appl. No.: 19/438,415

(22) Filed: Dec. 31, 2025

Related U.S. Application Data
(63) Continuation-in-part of application No. 19/253,600, filed on Jun. 27, 2025.
(63) Continuation-in-part of application No. 18/935,470, filed on Nov. 2, 2024.

(51) Int. Cl. A61N 5/06

(52) U.S. Cl. CPC A61N 5/0624; A61N 2005/0626; A61N 2005/0632; A61N 2005/0645; A61N 2005/0652; A61N 2005/0659; A61N 2005/0663

(57) ABSTRACT

A lower back wearable radiation emitting device with integrated vibration element which facilitates a provision of radiation treatment in conjunction with vibration treatment on the lower back of a wearer. The wearable radiation emitting device with integrated vibration element operates to emit a spectrum of near infrared light and infrared light suitable for promoting recovery and reduction of general pain as well as infection prevention in post op scenarios and rehabilitation scenarios for patients dealing with lower back injuries while also providing vibration therapy to the injury site which further helps with regulating swelling and stimulating the lymphatic system in the region. In this regard, the wearable radiation emitting device with integrated vibration element is specifically suited to assist the rehabilitation and healing process as well as pain management for patients who are dealing with lower back injuries.

### CROSS REFERENCE TO RELATED APPLICATIONS

This application is a continuation in part of, incorporates by reference, and claims the benefit of co-pending U.S. nonprovisional patent application Ser. No. 19/253,600, filed Jun. 27, 2025 and co-pending U.S. nonprovisional patent application Ser. No. 18/935,470, filed Nov. 2, 2024.

### BACKGROUND OF THE INVENTION

### Field of the Invention

This invention relates generally to wearable therapeutic devices and, more particularly, to a wearable device which provides for the application of therapeutic radiation at a plurality of wavelengths and vibration to the lower back of a wearer's body.

### Description of the Prior Art

Lower back injury due to medical illness, trauma or surgery often requires post injury or post operative treatment. Such treatments often take on various forms, and may include a combination of rehabilitation, pain management, infection prevention and repair of the skin at or around the site of the injury. Such treatment has been shown to improve the recovery success rate for the injury and promote the surgical site to heal correctly without secondary issues as well as improve the quality of the rehabilitation of non-surgical injuries

While there are various forms of post operative and rehabilitee treatment methods for lower back injuries, it has been found that light therapy, such as therapy in the visible light and infrared spectrums, can provide a range of benefits with relative ease of application suitable for a patent to conduct themselves or with assistance at various stages of the rehabilitation process. It has also been found that adding vibration therapy in conjunction with light therapy can further improve the quality and rate of the healing process and further support the rehabilitation efforts.

The combination of these treatment methods can be used on their own and do not require additional supporting treatment methods. Although, they can also be used in conjunction with any other rehab method desired, to try and maximize the benefits of the rehab therapy, making it a highly versatile way of supporting rehabilitation.

With respect to light therapy, the application of various specific spectrums of visible and infrared light has been shown to promote blood flow and oxygenation to the site and therefore increase the rate at which the site heals while reducing scarring. Light spectrums such as near infrared blue light have also been shown to reduce bacterial infection by killing unwanted bacteria such as Staphylococcus virus, which is a major risk factor in post-op scenarios. A key benefit of improved healing is that it can oftentimes reduce the amount of pain experienced by the patient. The effects of the various infrared light spectrums also reduce the likelihood of conditions such as phantom limb pain syndrome, which is often triggered by blood flow issues to the amputation site, excessive swelling or infection.

Vibration therapy can improve blood flow, which it can do by dilating blood vessels in the region of the body to which it is applied. It also stimulates the lymphatic system region of the body to which it is applied, which may be an injury site, which can result in an influx of antibodies to the injury site. Through this mechanism, vibration therapy can effectively reduce swelling as the lymphatic system can help prevent edema by regulating fluid levels around the injury site.

Accordingly, there remains a need for a radiation emitting device which provides light therapy by emitting radiation at desired wavelengths and which has an integrated vibration therapy element. It would be desirable for such a radiation emitting device with integrated vibration therapy element to be specifically designed for use by patients with lower back injuries by themselves or with minimal assistance at various stages of the rehabilitation process. Along these lines, it would additionally be desirable for such a radiation emitting device with integrated vibration therapy element to be easily fitted and comfortable to wear, and have a control interface which is reachable while worn so as to allow for easy operation of the device. Ideally, such a device would also incorporate the most effective spectrums of light to aid the recovery process.

### SUMMARY OF THE INVENTION

The present disclosure provides for a lower back wearable radiation emitting device with integrated vibration element, comprising: a device body having a first side and an opposing second side, wherein the device body is adapted to be wrapped around a wearer's torso adjacent to a target area on the wearer's torso; a plurality of electrical radiation emitting aspects positioned in an emitting section of the first side and configured to selectively generate radiation when activated, wherein the plurality of radiation emitting aspects are arranged such that when activated with the device body adjacent to the target area, the plurality of radiation emitting aspects direct radiation into the target area; at least one vibrating element adapted to selectively produce vibrational motion, wherein the at least one vibrating element is integrated with the device body such that such that when activated with the device body adjacent to the target area, motion produced by the at least one vibrating element is transferred the wearer's torso; and wherein the plurality of radiation emitting aspects include at least a first region of radiation emitting aspects and a second region of radiation emitting aspects, with the first region of radiation emitting aspects being more densely clustered than the second region of radiation emitting aspects.

It is an object of the present disclosure to provide a radiation emitting device which provides light therapy by emitting radiation at desired wavelengths and which has an integrated vibration therapy element.

It is an additional object of the present disclosure to provide a radiation emitting device with integrated vibration therapy element specifically designed for use by patients with lower back injuries by themselves or with minimal assistance at various stages of the rehabilitation process.

It is an additional object of the present disclosure to provide a radiation emitting device with integrated vibration therapy element that is easily fitted and comfortable to wear, and have a control interface which is reachable while worn so as to allow for easy operation of the device.

### BRIEF DESCRIPTION OF THE DRAWINGS

FIG. 1 is a front elevational view of a wearable radiation emitting device with integrated vibration element built in accordance with the present disclosure, shown in place on a user.

FIG. 2 is a rear elevational view of a wearable radiation emitting device with integrated vibration element built in accordance with the present disclosure, shown in place on a user.

FIG. 3 is a perspective view of a wearable radiation emitting device with integrated vibration element built in accordance with the present disclosure, showing a treatment side of a device body.

FIG. 4 is a perspective view of a wearable radiation emitting device with integrated vibration element built in accordance with the present disclosure, showing a control side of a device body with a detachable controller.

FIG. 5 is an exploded perspective view of a wearable radiation emitting device with integrated vibration element built in accordance with the present disclosure, showing the control side of the device body with the detachable controller removed.

FIG. 6 is a plan view of the detachable controller of a wearable radiation emitting device with integrated vibration element built in accordance with the present disclosure, showing a first side thereof.

FIG. 7a is a perspective view of a wearable radiation emitting device with integrated vibration element built in accordance with a single pad embodiment of the present disclosure, showing a control side of a device body with a detachable controller with the embedded vibration element represented.

FIG. 7b is a perspective view of a wearable radiation emitting device with integrated vibration element built in accordance with a dual pad embodiment of the present disclosure, showing a control side of a device body with a detachable controller with the embedded vibration element represented.

FIG. 8 is an exploded perspective view of a wearable radiation emitting device with integrated vibration element built in accordance with the present disclosure, showing the treatment side of the device body and removable cover.

FIG. 9 is a top plan view of a removable cover of a wearable radiation emitting device with integrated vibration element built in accordance with the present disclosure.

FIG. 10 is a graph showing an exemplary single wavelength output for LEDs in a wearable radiation emitting device with integrated vibration element built in accordance with the present disclosure.

FIG. 11 is a graph showing an exemplary multiple wavelength output for LEDs in a wearable radiation emitting device with integrated vibration element built in accordance with the present disclosure.

### DETAILED DESCRIPTION OF THE INVENTION

Described herein is a lower back wearable radiation emitting device with integrated vibration element which facilitates a provision of radiation treatment in conjunction with vibration treatment on the lower back of a wearer. The wearable radiation emitting device with integrated vibration element operates to emit a spectrum of near infrared light and infrared light suitable for promoting recovery and reduction of general pain as well as infection prevention in post op scenarios and rehabilitation scenarios for patients dealing with lower back injuries, while also providing vibration therapy to the injury site which further helps with stimulating the lymphatic system in the region and regulating swelling. In this regard, the wearable radiation emitting device with integrated vibration element is specifically suited to assist the rehabilitation and healing process as well as pain management for patients who are dealing with lower back injuries, which are common and can be brought on by lifestyle factors, muscular-skeletal imbalances, trauma due to sport or other impact related injury such as a fall or a motor vehicle accident or for post op scenarios.

It is appreciated that a key function of the lower back wearable radiation emitting device with integrated vibration element relates to the use of various spectrums of light to promote healing of the injury site and reduce pain through increased blood flow and oxygenation to the lower back, which improves the rate and quality at which the soft tissue heals. By achieving improved cellular health and reduced inflammation, the lower back wearable radiation emitting device with integrated vibration element can assist with reducing pain experienced from an injury or pinched nerve. Benefits also include infection management by killing bacteria such as Staphylococcus virus and other harmful bacteria on the injury site which would otherwise disrupt the healing and rehabilitation progress.

In addition to the benefits received by various spectrums of light, the lower back wearable radiation emitting device with integrated vibration element also utilizes vibration to further increase blood flow to the trauma site by dilating blood vessels which promotes faster healing. This vibration also stimulates the lymphatic system which further assists with preventing infection and leads to systemic healing, as well as reduces swelling by regulating fluid buildup in the region. Such benefits have been shown to lead to improved local and systemic healing and reduction of pain and swelling experienced by the patient during the rehabilitation process.

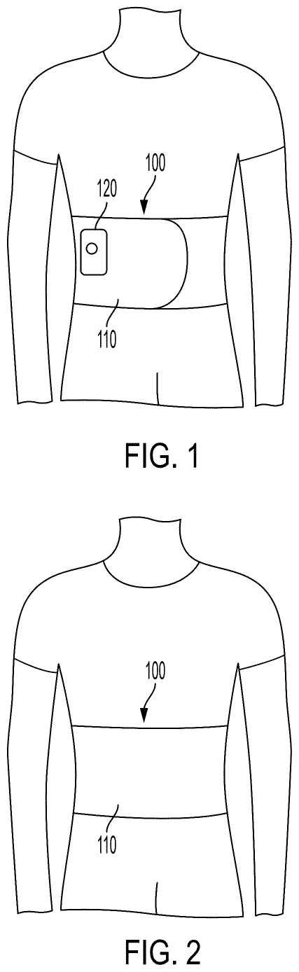

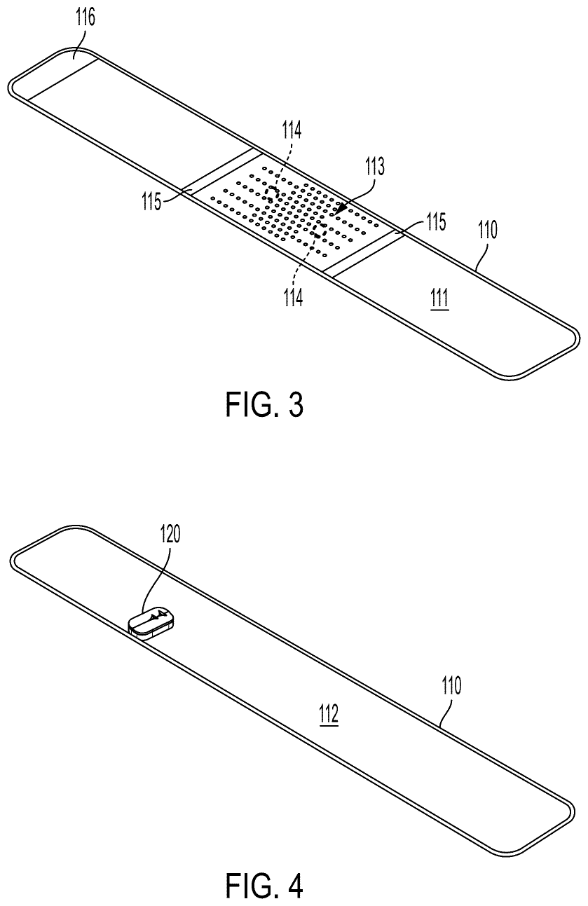

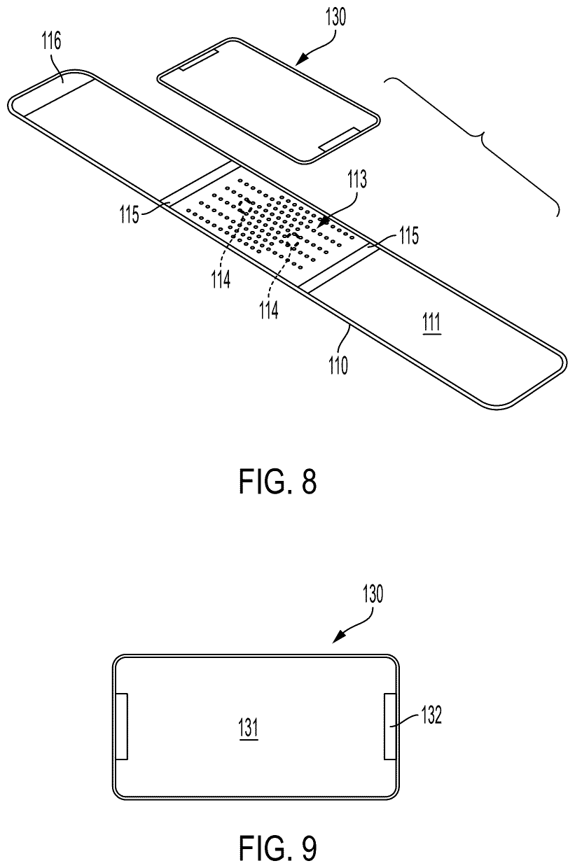

Referring now to the drawings and, in particular, FIGS. 1, 2, 3, 4, 5, 6, 7a, 7b, 8 and 9, a lower back wearable radiation emitting device with integrated vibration element 100 is shown as a brace body 110 and a control module 120. The brace body 110 may be defined by a substantially flat, elongated member, such as a wearable lower back wrap or lower back soft brace having a treatment side 111 and an opposing control side 112, and may be constructed of a nylon neoprene material that is padded. The brace body 110 may be edged with binding to protect the material from wear while preventing any unfinished edges from scratching against a wearer.

The treatment side 111 of the brace body 110 includes a plurality of LED lights 113 mounted to its surface and either one or a pair of vibrating pads 114 which may be defined as medical vibrating pads formed from electrical motors that may be positioned right below the outer layer of the surface of the treatment side 111. The LED lights 113 are centered on the treatment side 111 forming an emitting section of the treatment side 111 that is centered on the lower back of a wearer. The vibrating pad(s) 114 may also be located in the emitting section, positioned as discussed below. When in use, the LED lights 113 may be deployed in multiple groups, with each group having both infrared, red (visible) and blue (visible) LEDs in alternating rows to allow for full coverage of all required spectrums. In this regard, when the brace body 110 is fitted on a wearer, these LEDs are orientated so that they are positioned to direct radiation to the lower back area of a wearer so as to be suitable to provide light therapy.

The treatment side 111 may also include cover fastener sections 115 which allow for attachment of a protective cover 130 and a wrap fastener section 116 which allows the brace body 110 to be secured to itself and hold in place when wrapped around a wearer, as shown in FIGS. 1 and 2. The cover fastener sections 115 may run alongside the emitting section, while the wrap fastener section 116 may be disposed at one end of the brace body 110. The cover fastener sections 115 may be formed of a portion of a hook and loop fastener, such as a loop portion of a hook and loop fastener. The wrap fastener section 116 may be formed of a hook portion of a hook and loop fastener so that it is able to selectively couple with the control side 112 when the brace body 110 is wrapped around a wearer.

Embodiments of the brace body 110 may be formed with the emitting section of the treatment side 111 centrally positioned between two flanking sections on treatment side 111, with the flanking sections extending each extending from one end of the brace body 110 on the treatment side to the cover fastener section 115 that is closest to that end of the brace body 110. In some embodiments, the flanking sections may each be larger (i.e., be longitudinally longer) than the emitting section.

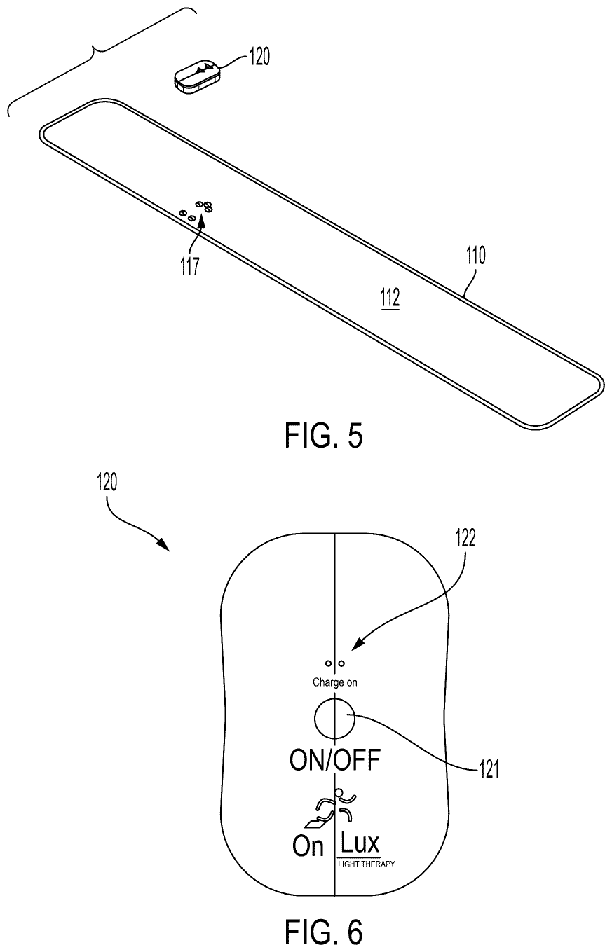

The control side 112 of the brace body 110 includes magnetic mounting interface 117, which allows for the removable attachment of the control module 120 to the brace body 110 and thus may be exposed when the control module 120 is not attached to the brace body 110. The magnetic mounting interface 113 may be constructed of or include a plurality of connectors constructed of a ferromagnetic material, arranged in a pattern such as what is shown in FIG. 5, and may be electrically connected to the LED lights 113 and the vibrating pad(s) 114. As such, the magnetic mounting interface 113, together with a magnetic coupler of the control module 120, allow for the control module 120 to be removably attached magnetically to the brace body 110 and for the transmission of electricity and electrical signals between the control module 120 and the LED lights 113 and the vibrating pad(s) 114.

In some embodiments, magnetic mounting interface 113 may also include, or be constructed as, a mechanical clip, such as a spring biased clip.

The control module 120 may be constructed of rigid housing made of injected plastic and may include therein a controller and a power source such as a rechargeable or replaceable battery, and include thereon a magnetic coupling interface (not shown), with each of these items electrically interconnected such that the controller can use power from the power source to generate electrical signals and send electrical signals it has generated through the magnetic coupler. The magnetic coupling interface may be formed of a plurality of coupling members constructed of a ferromagnetic material and arranged in a pattern that corresponds to the magnetic mounting interface 113 shown in FIG. 5. The housing may feature a minimalistic and sleek design to allow for comfortable use and wear, with rounded edges preventing any scratching or digging into a wearer.

The control module 120 includes an actuator button 121 or other manual actuators, with each button 121 electrically connected to the controller and being designated to control the operation of the LED lights 113 on the brace body 110 and the control the operation of the vibrating pad(s) 114 in the brace body 110. This button 121 may be labeled on the control module 120 for easy use.

The control module 120 may also include charge indicator lights 122 which are electrically connected to the controller and used to allow the controller to visually indicate whether battery level is low or sufficiently charged. These lights may be positioned near the top of the control module 120.

The battery in the control module 120 serves as the power source for the electrical components of the wearable radiation emitting device with integrated vibration element 100. Advantageously, having to disconnect the control module 120 for battery charging or replacement ensures that the device is not used during such times for safety purposes. Also, the brace body 110 is arranged with the control module 120 on the flip side as the treatment side 111, located away from the central area of the brace body 110 so as to prevent cases where fluid can come in contact with the control module 120 while the wearable radiation emitting device with integrated vibration element 100 is being worn. Moreover, this allows for the control module 120 to be positioned in front of a wearer when it is being worn (as seen in FIG. 1) so as to remain easily accessible, with its charge indicator lights visible, when the wearable radiation emitting device with integrated vibration element 100 is in place on a wearer. This allows the wearer to operate the wearable radiation emitting device with integrated vibration element 100 by turning it on or off without the need to remove it or strain in order to reach it. The design also allows for sufficient airflow to prevent excess perspiration and heat build-up.

The LED lights 113 on the treatment side 111 may be grouped into defined treatment regions, with the placement of treatment regions including an intense area with densely clustered LEDs focused on the lower spine and erector muscles of the lower back. The treatment regions may also include moderate areas where LEDs are less densely clustered on the sides of and moving further away from the intense area. This operates to ensure that the area being treated is not only the immediate region of the lower spine and erector muscles of the lower back but the area around the lower spine and erector muscles of the lower back. In other words, the LED lights 113 are arranged together such that when activated with the treatment side 111 of the brace body 110, adjacent to a target area defined by the lower spine and erector muscles of the lower back, the LED lights 113 will direct radiation into one or more other secondary areas which are adjacent to the target area, in addition to the target area, thereby providing a more holistic treatment for the wearer and further reducing any issues surrounding the area. Such an approach can assist greatly with reducing overall inflammation and swelling.

The LED lights 113 may be made up of a first group of LED bulbs which emit a first type of radiation and a second group of LED bulbs which emit radiation at one or more other spectrums. For example, first group of LED bulbs may emit a 470 nm wavelength blue light in the visible spectrum, while the second group of LED bulbs may emit infrared radiation and the second group may emit radiation a 660 nm red light in the visible spectrum, a 830 nm infrared light, and a 950 nm infrared light. The first group and the second group of LEDs may be placed in an alternating pattern so that the various wavelengths of light are evenly distributed to the treatment region.

With respect to the vibrating pad(s) 114, it is contemplated that the vibrating pad(s) 114 may be positioned in such a way that when the lower back wearable radiation emitting device with integrated vibration element 100 is in place on a wearer, the vibrating pad(s) 114 will stimulate the lymphatic system of the wearer around the trauma site (or otherwise the area being treated). In this regard, the vibrating pad(s) 114 may be positioned so as to be directly over the spine of a wearer (in a single pad embodiment) or positioned so as to be on either side of the spine of the wearer (in a dual pad embodiment). It is appreciated that whether the single pad embodiment or the dual pad embodiment will be used may depend on the type of injury/condition being treated and the requirements of the targeted region of the wearers back being targeted for the vibration therapy.

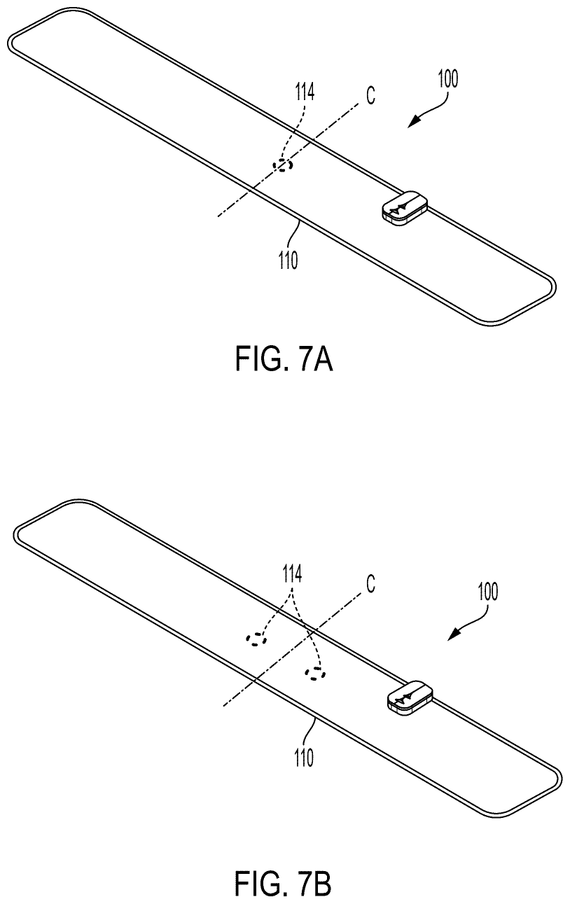

In the single pad embodiment, the single vibrating pad 114 may be centrally positioned in the brace body 110 such that a bisecting line C representing the exact center of the brace body 110 would pass through it, as illustrated in FIG. 7a. In this regard, when the lower back wearable radiation emitting device with integrated vibration element 100 built in accordance with the single pad embodiment is worn, the single vibrating pad 114 may be positioned directly on the spine of a wearer. In this embodiment, it is contemplated that the vibrating pad 114 is disposed right in the middle of the intense area treatment region of the lower back wearable radiation emitting device with integrated vibration element 100.

In the dual pad embodiment, the two vibrating pads 114 may be positioned in the brace body 110 in mirror image locations, with each being set off from a bisecting line C representing the exact center of the brace body 110 by seventy (70) millimeters (and thus one hundred and forty millimeters from each other), as illustrated in FIG. 7b. In this regard, when the lower back wearable radiation emitting device with integrated vibration element 100 built in accordance with the dual pad embodiment is worn, the one of the vibrating pads 114 may be positioned on either side of the spine of the wearer. Such positioning may result in the vibrating pads 114 of the dual pad embodiment may be positioned on either side of the intense area treatment region, just where the moderate area treatment region begins. This allows the vibrating pads 114 to be located on either side of the spine, as well as on the sides of the waist. This also causes the vibrating pads 114 to give complete coverage to the area being treated, and to affect the lymphatic system in the area being treated as well as adjacent areas.

The brace body 110 may also be used with a protective cover 130, with the protective cover 130 being formed from a transparent plastic screen having binding 131 on the edges to prevent scratching a wearer and allow for a uniform finish. The protective cover 130 may operate to prevent fluid or leakage from contaminating the device when used on a wearer with open wounds or on a recently operated region while allowing light to pass through. The protective cover 130 may include protector fastener 132 defined by a portion of a hook and loop fastener which corresponds to the portion of the hook and loop fastener which define the cover fastener sections 115, so as to allow for the removable attachment of the protective cover 130 to the brace body 110. In this regard, not only can the protective cover 130 protect the LED lights 113 and the vibrating pads 114, it also can be removed for cleaning in between uses.

It is appreciated that if a wearer's treatment site has no open wound, then the brace body 110 may be worn without the protective cover 130.

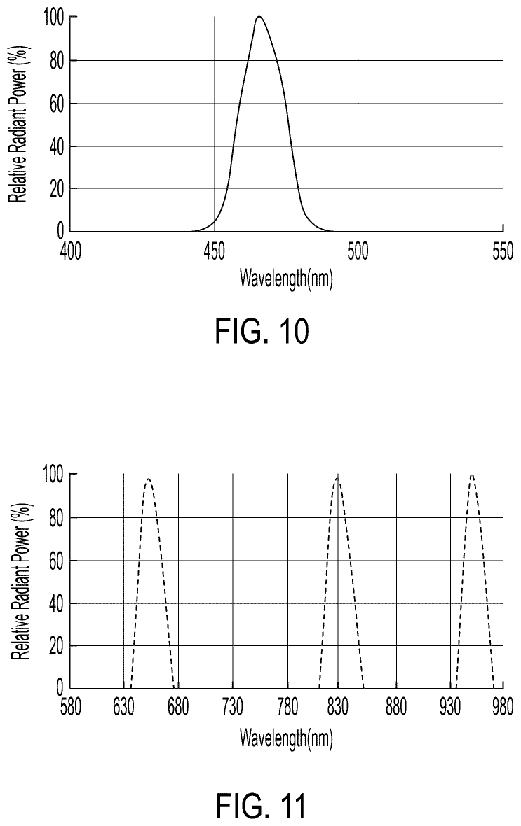

Referring now to FIG. 10, the wavelength emitted by the 470 nm blue LED light when operating at full relative radiant power is indicated. As is evident, the wavelength varies from around 450 nm to 490 nm as the light is operated. When operating at full capacity as it would be on the wearable radiation emitting therapy device, it emits light in the 470 nm wavelength spectrum.

Referring now to FIG. 11, the three-in-one LED light emits a spectrum around three target wavelengths. The exact wavelengths are achieved when the LED light is operating at full relative radiant power, as shown. These wavelengths are 660 nm (red light), 830 nm (infrared light), and 950 nm (infrared light), respectively. It is contemplated, however, that these wavelengths may vary by roughly 20 nm in either direction as the LED lights reach their full power, where the exact frequency is achieved.

It is appreciated that LED light therapy, particularly using a variety of wavelengths of light, assists in various ways, such as promoting blood circulation, which in turn improves oxygenation. It also assists in detoxification and an influx of antibodies to the region. Other benefits include improved scar healing and general skin healing through increased collagen production. Furthermore, as certain wavelengths have antibacterial properties, such that the blue light emits, these assists in killing bacteria including Staphylococcus and other skin bacteria.

For example, the infrared light is able to penetrate deep into the skin and tissue and promote blood flow and therefore provide improved oxygenation as well as a greater influx of antibodies to the site. This phenomenon, in conjunction with the increased collagen production, results in improved skin repair and cellular regeneration, boosts new cell growth, enhances skin rejuvenation and stimulates various cellular processes and increases rejuvenation.

The blue light has similar benefits to infrared light with regards to increasing blood flow and its associated benefits. In addition, blue LED light therapy stimulates fibroblasts, the cells responsible for collagen production in the skin. This can help reduce the appearance of fine lines, wrinkles, and even scars. Advantageously, blue light has also been shown to have the most effective antimicrobial spectral range. This assists in killing various bacteria and fungi, allowing the skin to heal without secondary ailments such as fungal infection, acne and infections such as Staphylococcus. Blue light has also been shown to suppress cells that contribute to inflammation in the skin, which can also treat chronic inflammatory skin conditions such as eczema.

The instant disclosure employs vibration therapy synergistically with the radiation treatment. The vibrating pads, which are used to provide for the vibration therapy, may be placed in such a way that they are in the correct regions to stimulate the lymphatic system around the treatment area. These pads may be set to vibrate at a frequency which optimizes the stimulation of the lymphatic system and the increase in blood flow, such as at 13000 RPM, +/−3000, with a twenty-five (25) second on and five (5) second off cycle.

It is appreciated that the lymphatic system assists in defending the body against infection, which is important to keep trauma sites healthy during recovery. Stimulating the lymphatic system can also lead to systemic healing. Along these lines, the wearable radiation emitting device with integrated vibration element incorporates two motors which provide the vibration functionality, fitted on either side of the spine in the lower back region, when the device is fitted to the patient. In alternate embodiments, however, more than two motors may be employed.

The lymphatic system is also responsible for regulating fluid levels in the body. This is especially important when dealing with soft tissue damage as patients often experience a fluid buildup at the trauma site when dealing with soft tissue injuries. As such, stimulating the lymphatic system in these regions will assist in reducing the swelling by transporting the excess fluid away from the injury site. While doing so it will also filter out waste or abnormal cells from this fluid, helping to keep the body healthy.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A lower back wearable radiation emitting device with integrated vibration element, comprising:
a device body having a first side and an opposing second side, wherein the device body is elongated and adapted to be wrapped around a wearer's torso adjacent to a target area on the wearer's torso;
a plurality of electrical radiation emitting aspects positioned in an emitting section of the first side and configured to selectively generate radiation when activated, wherein the plurality of radiation emitting aspects are arranged such that when activated with the device body adjacent to the target area, the plurality of radiation emitting aspects direct radiation into the target area;
at least one vibrating element adapted to selectively produce vibrational motion, wherein the at least one vibrating element is integrated with the device body such that such that when activated with the device body adjacent to the target area, motion produced by the at least one vibrating element is transferred the wearer's torso; and
wherein the plurality of radiation emitting aspects include at least a first region of radiation emitting aspects and a second region of radiation emitting aspects, with the first region of radiation emitting aspects being more densely clustered than the second region of radiation emitting aspects.

2. The lower back wearable radiation emitting device with integrated vibration element of claim 1, wherein the plurality of radiation emitting aspects are defined by a set of LED lights which collectively are configured to selectively generate radiation at a plurality of wavelengths when activated.

3. The lower back wearable radiation emitting device with integrated vibration element of claim 1, the first region of radiation emitting aspects and the second region of radiation emitting aspects each include at least one discrete radiation emitting aspect which generates radiation at a first wavelength when activated and at least one discrete radiation emitting aspect which generates radiation at a second wavelength when activated.

4. The lower back wearable radiation emitting device with integrated vibration element of claim 1, wherein the emitting section of the first side is centrally positioned between two flanking sections of the first side.

5. The lower back wearable radiation emitting device with integrated vibration element of claim 1, additionally comprising a control module operative to selectively activate the electrical radiation emitting aspects and the at least one vibrating element, wherein the control module is removably attached to the second side the control module and device body are configured such that electrical operating power is only supplied to the electrical radiation emitting aspects and the at least one vibrating element when the control module is attached to the second side.

6. The lower back wearable radiation emitting device with integrated vibration element of claim 1, wherein the at least one vibrating element is centrally positioned longitudinally on the device body.

7. The lower back wearable radiation emitting device with integrated vibration element of claim 1, wherein the at least one vibrating element is integrated with the device body such that such that when activated with the device body adjacent to the target area, motion produced by the at least one vibrating element is transferred to at least the target area.

8. The lower back wearable radiation emitting device with integrated vibration element of claim 1, wherein the plurality of radiation emitting aspects are arranged such that when activated with the device body adjacent to the target area, the plurality of radiation emitting aspects direct radiation into at least one secondary area adjacent to the target area in addition to the target area.

9. The lower back wearable radiation emitting device with integrated vibration element of claim 1, additionally comprising a protective cover sized to be placed on and completely cover the first side and be removably attached to the first side.

10. A lower back wearable radiation emitting device with integrated vibration element, comprising:
a device body having a first side and an opposing second side, wherein the device body is elongated and adapted to be positioned on a wearer's torso adjacent to a target area on the wearer's torso;
a plurality of electrical radiation emitting aspects positioned in an emitting section of the first side and configured to selectively generate radiation when activated, wherein the plurality of radiation emitting aspects are arranged such that when activated with the device body adjacent to the target area, the plurality of radiation emitting aspects direct radiation into the target area;
wherein the emitting section of the first side is positioned between two flanking sections of the first side;
at least one vibrating element adapted to selectively produce vibrational motion, wherein the at least one vibrating element is integrated with the device body such that such that when activated with the device body adjacent to the target area, motion produced by the at least one vibrating element is transferred the wearer's torso;
a control module operative to removably attach to the device body and selectively activate the radiation emitting aspects and the at least one vibrating element, wherein the control module and device body are configured such that electrical operating power is only supplied to the radiation emitting aspects and the at least one vibrating element when the control module is attached to the device body; and
a protective cover sized to be placed on and completely cover the first size and be removably attached to the first size.

11. The lower back wearable radiation emitting device with integrated vibration element of claim 10, wherein the plurality of radiation emitting aspects are defined by at least a first discrete set of LED lights and a second discrete set of LED lights, with the first discrete set of LED lights being operative to selectively generate radiation at a first wavelength when activated and the second discrete set of LED lights being operative to selectively generate radiation at least one wavelength that is not the first wavelength when activated.

12. The lower back wearable radiation emitting device with integrated vibration element of claim 11, wherein:
the plurality of radiation emitting aspects include at least a first region of radiation emitting aspects and a second region of radiation emitting aspects, with the first region of radiation emitting aspects being more densely clustered than the second region of radiation emitting aspects; and
the first discrete set of LED lights and the second discrete set of LED lights are positioned in an alternating pattern in both the first region of radiation emitting aspects and the second region of radiation emitting aspects.

13. The lower back wearable radiation emitting device with integrated vibration element of claim 10, wherein the device body and control module are configured such that the control module removably attaches to the second side.

14. The lower back wearable radiation emitting device with integrated vibration element of claim 10, wherein:
the at least one vibrating element is centrally positioned longitudinally on the device body; and
the at least one vibrating element is integrated with the device body such that when activated with the device body adjacent to the target area, motion produced by the at least one vibrating element is transferred to at least the target area.

15. The lower back wearable radiation emitting device with integrated vibration element of claim 10, wherein the plurality of radiation emitting aspects are arranged such that when activated with the device body adjacent to the target area, the plurality of radiation emitting aspects direct radiation into at least one secondary area adjacent to the target area in addition to the target area.

16. The lower back wearable radiation emitting device with integrated vibration element of claim 10, the at least one vibrating element defines a pair of vibrating elements, wherein the pair of vibrating elements are each positioned on the device body an equal distance from a longitudinal center of the device body.

17. A lower back wearable radiation emitting device with integrated vibration element, comprising:
a device body having a first side and an opposing second side, wherein the device body is elongated and adapted to be positioned on a wearer's torso adjacent to a target area on the wearer's torso;
a plurality of electrical radiation emitting aspects positioned in an emitting section of the first side and configured to selectively generate radiation when activated, wherein the plurality of radiation emitting aspects are arranged such that when activated with the device body adjacent to the target area, the plurality of radiation emitting aspects direct radiation into the target area;
wherein the emitting section of the first side is centrally positioned between two flanking sections of the first side;
wherein the plurality of radiation emitting aspects include at least a first region of radiation emitting aspects and a second region of radiation emitting aspects, with the first region of radiation emitting aspects being more densely clustered than the second region of radiation emitting aspects;
a pair of vibrating elements adapted to selectively produce vibrational motion, wherein the pair of vibrating elements are integrated with the device body such that such that when activated with the device body adjacent to the target area, motion produced by the pair of vibrating elements is transferred the at least the target area;
wherein the pair of vibrating elements are each positioned on the device body an equal distance from a longitudinal center of the device body; and
a control module operative to removably attach to the second side and selectively activate the radiation emitting aspects and the at least one vibrating element, wherein the control module and device body are configured such that electrical operating power is only supplied to the radiation emitting aspects and the at least one vibrating element when the control module is attached to the second side.

18. The lower back wearable radiation emitting device with integrated vibration element of claim 17, wherein the plurality of radiation emitting aspects are defined by at least a first discrete set of LED lights and a second discrete set of LED lights, with the first discrete set of LED lights being operative to selectively generate radiation at a first wavelength when activated and the second discrete set of LED lights being operative to selectively generate radiation at least one wavelength that is not the first wavelength when activated.

19. The lower back wearable radiation emitting device with integrated vibration element of claim 18, wherein:
the first discrete set of LED lights and the second discrete set of LED lights are positioned in an alternating pattern in both the first region of radiation emitting aspects and the second region of radiation emitting aspects; and
the plurality of radiation emitting aspects are arranged such that when activated with the device body adjacent to the target area, the plurality of radiation emitting aspects direct radiation into at least one secondary area adjacent to the target area in addition to the target area.

20. The lower back wearable radiation emitting device with integrated vibration element of claim 19, additionally comprising a protective cover sized to be placed on and completely cover the first side and be removably attached to the first side.
